SEQUENCE LISTING (45 sequences)

SEQ ID NO: 1 - DNA, 106 bp, Homo sapiens
tatgtgcggttctggttctgacggtggtgtttgcccgaagatcttgaaaaaatgccgtcgtgactctgactgcccgggtgcttgcatctgccgtggtaacggttac

SEQ ID NO: 2 - DNA, 107 bp, Artificial Sequence
  anti-sense oligonucleotide
gcagtaaccgttaccacggcagatgcaagcacccgggcagtcagagtcacgacggcattttttcaagatcttcgggcaaacaccaccgtcagaaccagaaccgcaca

SEQ ID NO: 3 - protein, 35 aa, Homo sapiens
MCGSGSDGGVCPKILKKCRRDSDCPGACICRGNGY

SEQ ID NO: 4 - protein, 11 aa, Homo sapiens
FTALDWSWLQT

SEQ ID NO: 5 - DNA, 48 bp, Artificial Sequence
  sense oligonucleotide
tatgtgctttaccgcgctggattggagctggctgcagacctgcccgaa

SEQ ID NO: 6 - DNA, 50 bp, Artificial Sequence
  anti-sense oligonucleotide
gatcttcgggcaggtctgcagccagctccaatccagcgcggtaaagcaca

SEQ ID NO: 7 - protein, 17 aa, Artificial Sequence
  Synthetic peptide
MCFTALDWSWLQTCPKI

SEQ ID NO: 8 - DNA, 113 bp, Artificial Sequence
  Synthetic oligonucleotide
atgtgctttaccgcgctggattggagctggctgcagacctgcccgaagatcttgaaaaaatgccgtcgtgactctgactgcccgggtgcttgcatctgccgtggtaacggtta

SEQ ID NO: 9 - protein, 38 aa, Artificial Sequence
  Synthetic peptide
MCFTALDWSWLQTCPKILKKCRRDSDCPGACICRGNGY

SEQ ID NO: 10 - protein, 37 aa, Artificial Sequence
  Synthetic peptide
FTALDWSWLQTCPKILKKCRRDSDCPGACICRGNGYC

SEQ ID NO: 11 - protein, 32 aa, Artificial Sequence
  Synthetic peptide
LDWSWLCPKILKKCRRDSDCPGACICRGNGYC

SEQ ID NO: 12 - protein, 35 aa, Artificial Sequence
  Synthetic peptide
FTALDWSWLCPKILKKCRRDSDCPGACICRGNGYC

SEQ ID NO: 13 - protein, 37 aa, Artificial Sequence
  Synthetic peptide
CFTALDWSWLQTCPKILKKCRRDSDCPGACICRGNGY

SEQ ID NO: 14 - protein, 32 aa, Artificial Sequence
  Synthetic peptide
CLDWSWLCPKILKKCRRDSDCPGACICRGNGY

SEQ ID NO: 15 - protein, 35 aa, Artificial Sequence
  Synthetic peptide
CFTALDWSWLCPKILKKCRRDSDCPGACICRGNGY

SEQ ID NO: 16 - protein, 43 aa, Artificial Sequence
  Synthetic peptide
GGSFTALDWSWLQTGGSCPKILKKCRRDSDCPGACICRGNGYC

SEQ ID NO: 17 - protein, 49 aa, Artificial Sequence
  Synthetic peptide
GGSGGSFTALDWSWLQTGGSGGSCPKILKKCRRDSDCPGACICRGNGYC

SEQ ID NO: 18 - protein, 55 aa, Artificial Sequence
  Synthetic peptide
GGSGGSGGSFTALDWSWLQTGGSGGSGGSCPKILKKCRRDSDCPGACICRGNGYC

SEQ ID NO: 19 - protein, 43 aa, Artificial Sequence
  Synthetic peptide
CGGSFTALDWSWLQTGGSCPKILKKCRRDSDCPGACICRGNGY

SEQ ID NO: 20 - protein, 49 aa, Artificial Sequence
  Synthetic peptide
CGGSGGSFTALDWSWLQTGGSGGSCPKILKKCRRDSDCPGACICRGNGY

SEQ ID NO: 21 - protein, 55 aa, Artificial Sequence
  Synthetic peptide
CGGSGGSGGSFTALDWSWLQTGGSGGSGGSCPKILKKCRRDSDCPGACICRGNGY

SEQ ID NO: 22 - protein, 6 aa, Artificial Sequence
  Synthetic peptide
LDWSWL

SEQ ID NO: 23 - protein, 9 aa, Artificial Sequence
  Synthetic peptide
FTALDWSWL

SEQ ID NO: 24 - protein, 34 aa, Artificial Sequence
  Synthetic peptide
CGSGSDGGVCPKILQRCRRDSDCPGACICRGNGY

SEQ ID NO: 25 - protein, 34 aa, Artificial Sequence
  Synthetic peptide
CGSGSDGGVCPKILXXCRRDSDCPGACICRGNGY

SEQ ID NO: 26 - protein, 37 aa, Artificial Sequence
  Synthetic peptide
FTALDWSWLQTCPKILXXCRRDSDCPGACICRGNGYC

SEQ ID NO: 27 - protein, 8 aa, Artificial Sequence
  Synthetic peptide
GSGSDGGV

SEQ ID NO: 28 - protein, 37 aa, Artificial Sequence; identical to SEQ ID NO: 10
  Synthetic peptide

SEQ ID NO: 29 - protein, 38 aa, Artificial Sequence
  Synthetic peptide
FTALDWSWLQTCPKILKKCRRDSDCPGACICRGNGYCG

SEQ ID NO: 30 - protein, 40 aa, Artificial Sequence
  Synthetic peptide
GVCPKILKKCRRDSDCPGACICRGNGYCTQLWSWDLATFG

SEQ ID NO: 31 - protein, 38 aa, Artificial Sequence
  Synthetic peptide
TQLWSWDLATFCPKILKKCRRDSDCPGACICRGNGYCG

SEQ ID NO: 32 - protein, 40 aa, Artificial Sequence
  Synthetic peptide
GVCPKILKKCRRDSDCPGACICRGNGYCFTALDWSWLQTG

SEQ ID NO: 33 - protein, 49 aa, Artificial Sequence
  Synthetic peptide
FTALDWSWLQTCPKILKKCRRDSDCPGACICRGNGYCTQLWSWDLATFG

SEQ ID NO: 34 - protein, 49 aa, Artificial Sequence
  Synthetic peptide
TQLWSWDLATFCPKILKKCRRDSDCPGACICRGNGYCFTALDWSWLQTG

SEQ ID NO: 35 - protein, 51 aa, Artificial Sequence
  cyclized to N terminal glycine
GGVCPKILKKCRRDSDCPGACICRGNGYCGSGSGASKAPFTALDWSWLQTA

SEQ ID NO: 36 - protein, 51 aa, Synthetic peptide
  cyclized to N terminal Gly
GGVCPKILKKCRRDSDCPGACICRGNGYCGSGSGASKAPTQLWSWDLATFA

SEQ ID NO: 37 - protein, 51 aa, Artificial Sequence
  cyclized to N-terminal Gly
GVCPKIASKAPFTALDWSWLQTALKKCRRDSDCPGACICRGNGYCGSGSDG

SEQ ID NO: 38 - protein, 51 aa, Artificial Sequence
  cyclized to N-terminal Gly
GVCPKIASKAPTQLWSWDLATFALKKCRRDSDCPGACICRGNGYCGSGSDG

SEQ ID NO: 39 - protein, 51 aa, Artificial Sequence
  cyclized to N-terminal Gly
GVCPKILKKCRRDASKAPFTALDWSWLQTASDCPGACICRGNGYCGSGSDG

SEQ ID NO: 40 - protein, 51 aa, Artificial Sequence
  cyclized to N-terminal Gly
GVCPKILKKCRRDASKAPTQLWSWDLATFASDCPGACICRGNGYCGSGSDG

SEQ ID NO: 41 - protein, 51 aa, Artificial Sequence
  cyclized to N-terminal Gly
GVCPKILKKCRRDSDCPGASKAPFTALDWSWLQTAACICRGNGYCGSGSDG

SEQ ID NO: 42 - protein, 51 aa, Artificial Sequence
  cyclized to N-terminal Gly
GVCPKILKKCRRDSDCPGASKAPTQLWSWDLATFAACICRGNGYCGSGSDG

SEQ ID NO: 43 - protein, 50 aa, Artificial Sequence
  cyclized to N-terminal Gly
GVCPKILKKCRRDSDCPGACICRGNASKAPFTALDWSWLQTGYCGSGSDG

SEQ ID NO: 44 - protein, 50 aa, Artificial Sequence
  cyclized to N-terminal Gly
GVCPKILKKCRRDSDCPGACICRGNASKAPTQLWSWDLATFGYCGSGSDG

SEQ ID NO: 45 - protein, 11 aa, Artificial Sequence
  Synthetic peptide
TQLWSWDLATF

(19) United States
(12) Patent Application Publication
(10) Pub. No.: US 2015/0031598 A1
(43) Pub. Date: Jan. 29, 2015

(54) NEMO BINDING DOMAIN FUSION PROTEINS

(71) Applicant: Encode Bio, Inc., Los Angeles, CA (US)

(72) Inventors: Nima Shiva, Los Angeles, CA (US); Mark W. Nowak, Pasadena, CA (US)

(21) Appl. No.: 14/466,807

(22) Filed: Aug. 22, 2014

Related U.S. Application Data
(63) Continuation-in-part of application No. PCT/US2013/027702, filed on Feb. 25, 2013.
(60) Provisional application No. 61/869,030, filed on Aug. 22, 2013.
(60) Provisional application No. 61/602,264, filed on Feb. 23, 2012.

(51) Int. Cl. C07K 14/47; C07K 7/06

(52) U.S. Cl. CPC C07K 14/4703; C07K 7/06; C07K 2319/00
USPC 514/1.4; 530/324; 514/21.3; 514/15.1; 514/19.3

(57) ABSTRACT

Novel fusion proteins compromising nuclear factor kB essential modulator-binding (NEMO) domain or a fragment thereof and MCoTI-I/II or a fragment thereof, and their use for the treatment of inflammatory diseases and other medical conditions.

### CROSS-REFERENCE TO RELATED APPLICATIONS

This application is a continuation-in-part of, and claims the benefit of priority under 35 U.S.C. §120 from, the following U.S. patent applications: International Patent Application No. PCT/US2013/027702, filed Feb. 25, 2013 and entitled “NEMO BINDING DOMAIN FUSION PROTEINS;” U.S. Patent Application No. 61/602,264, filed on Feb. 23, 2012 and entitled, “NEMO BINDING DOMAIN FUSION PROTEINS;” and U.S. Patent Application No. 61/869,030, filed on Aug. 22, 2013 and entitled “NEMO BINDING DOMAIN FUSION PROTEINS.” The entire disclosure of the foregoing applications is incorporated by reference herein in its entirety.

### SEQUENCE LISTING

The entire content of a Sequence Listing titled “Sequence_Listing.txt,” created on Aug. 22, 2014 and having a size of 20 kilobytes, which has been submitted in electronic form in connection with the present application, is hereby incorporated by reference herein in its entirety.

### BACKGROUND

The nuclear factor kB (NF-κβ) essential modulator-binding domain (NEMO binding domain or NBD) blocks the activation of the IkB kinase (IKK) complex. NBD has been shown to selectively block the inflammation-induced activation of NF-κB when introduced into a cell. In view of this, NBD has been investigated as a therapeutic.

In order for NBD to be effective, however, it needs to be delivered to cells. Cell penetrating peptides have therefore been used as delivery vehicles to facilitate the introduction of NBD into cells. Such peptides, however, have been found to have a very narrow therapeutic range (i.e., having little difference between toxic and therapeutic doses), which severely limits their application as therapeutics. In anti-inflammatory animal models, known NBD peptides begin to demonstrate efficacy with doses ranging from 1-20 mg/kg, but toxicity has been observed in mice at doses greater than 50 mg/kg. This toxicity appears to be caused by high concentration of cell penetrating peptides (CPPs) used to deliver such NBD peptides, as similar toxicity is observed with an inactive mutant NBD peptide or when the CPPs alone are delivered. Accordingly, there is a need for potent anti-inflammatory molecules having a larger therapeutic range.

### SUMMARY

The fusion proteins disclosed comprise NBD or a fragment thereof and MCoTI-I/II or a fragment thereof. Preferably, the fusion protein is formed by replacing loop six of MCoTI-I/II with NBD or a fragment thereof, though NBD can also be fused to other portions of the MCoTI-I/II molecule. NBD can be represented by SEQ ID NOS. 4, 22, or 23, for example, while the fusion protein can be represented by SEQ ID NOS. 9, 10, 13-21, and/or 28-44. Preferably, the NBD or a fragment thereof is at an N-terminus of the fusion protein and replaces loop 6 of MCoTI-I/II. The fusion protein can be cyclic or linear, and can additionally compromise one or more linkers. It can also be formulated as a pharmaceutical composition in a pharmaceutically acceptable carrier.

The presently disclosed fusion proteins can be used to treat inflammatory diseases and other medical conditions of a subject. In one embodiment, a pharmaceutical composition compromising an NBD-MCoTI-I/II fusion protein can be administered to a subject in need thereof in a therapeutically effective amount.

### FIGURES

FIG. 1 is an illustration of the structure of MCoTI-I and MCoTI-II.

FIG. 2 is a graph showing that NBD-MCoTI-II inhibits NF-κβ signaling with greater potency than NBD Peptide.

FIG. 3 includes two graphs showing the dose-response relationship for NBD-MCoTI-II (graph A) and NBD peptide (graph B) inhibition of NF-κβ signaling in a cell-based assay. NBD-MCoTI-II is 10⁶-fold more potent than NBD peptide in this assay.

FIG. 4 is a graph showing that NBD-MCoTI-II reduces inflammation in a mouse CPE model.

FIG. 5 is another graph showing that NBD-MCoTI-II reduces inflammation in a mouse CPE model. NBD-MCoTI-II is 5000-fold more potent than NBD Peptide.

FIG. 6 is a graph showing that NBD-MCoTI-II reduces arthritic scores in a mouse CIA model.

FIG. 7 is a graph showing that NBD-MCoTI-II reduces paw swelling to background levels in a mouse CIA model.

FIG. 8 is a graph showing that NBD-MCoTI-II prevents arthritic damage in joints in a mouse CIA model.

FIG. 9 is a graph showing that NBD-MCoTI-II shows a remarkable reduction in composite histopathology score as compared to existing anti-arthritic drugs.

FIG. 10 sets forth sequences of nucleotides and polypeptides comprising NBD and MCoTI-II, with NBD sequences underlined.

FIG. 11 sets forth sequences of polypeptides comprising NBD and MCoTI-II, with NBD sequences underlined.

### DETAILED DESCRIPTION

### Definitions

As used herein, the following terms and variations thereof have the meanings given below, unless a different meaning is clearly intended by the context in which such term is used.

“About” when used in reference to a numerical value means plus or minus ten percent of the indicated amount. For example and not by way of limitation, “about 10” means between 9 and 11, and “about 10%” means between 9% and 11%.

“Amino acid” refers to natural amino acids as well as amino acid analogs (i.e., non-natural, synthetic, and modified amino acids, including D and L optical isomers).

“Bioactive peptide” refers to a peptide, such as NBD, having a pharmaceutical effect. Bioactive peptides may be peptide mimetics. Bioactive peptides may be proteins synthesized in the cell in the form of prepropeptides, which are then cleaved and modified to give active products. Bioactive peptides can be peptides with hormone- or drug-like activity that eventually modulate physiological function through binding interactions to specific receptors on target cells leading to induction of physiological responses. According to their functional properties, bioactive peptides can be classified as antimicrobial, antithrombotic, antihypertensive, opioid, immunomodulatory, mineral binding, and antioxidative, and the like.

“Cell penetrating peptides” (CPPs) refer to polypeptides which are able to translocate across a cell's plasma membrane, together with other moieties. Cell penetrating peptides are generally short peptides that facilitate cellular uptake of other molecules, in particular for therapeutic purposes. CPP's can be associated with the molecules being delivered either through chemical linkage via covalent bonds or through non-covalent interactions.

“Cyclic peptides” are polypeptide chains whose amino and carboxyl termini are themselves linked together with a peptide bond that forms a circular chain.

“Fragment” refers to a molecule, in particular an amino acid molecule, having truncations, deletions, and/or modifications with respect to a parent or wild-type molecule. Modifications of amino acid residues/proteins are well known in the art, and include, for example, acylation, methylation, phosphorylation, sulfation, and the like.

“Fused” and “fusion,” with respect to polypeptides, refers to a polypeptide formed by joining two or more precursor polypeptides. These polypeptides can be joined directly together, or other amino acid residues (i.e., linkers) can be situated between them. As such, polypeptides that are fused together may be connected directly or indirectly (i.e., via a linker or the like). As used herein, “fusion protein” refers primarily to the combination of NBD or a fragment thereof, and MCoTI-I/II or a fragment thereof. It is to be understood that the present fusion proteins can be produced by any means, such as via bacterial synthesis or automated synthesis.

“Linear,” in regard to polypeptides, refers to a polypeptide comprising a single linear polymer chain of amino acids bonded together. Linear polypeptides can be formed using automated synthesizers, for example, or through recombinant methods. Linear polypeptides may include disulfide bonds.

“Loop 6” refers to loop 6 of the MCoTI-I/II protein (SEQ ID NO:27).

“Medical condition” refers to both a disease and the symptoms of the disease.

“MCoTI-I/II” refers to a molecule or fragment thereof comprising SEQ ID NO:25 or a portion thereof. Amino acid 15 in this sequence can be either lysine or glutamine, and amino acid 16 can be either lysine or arginine. In wild-type MCoTI-I, amino acids 15 and 16 are glutamine and arginine, respectively, while in wild-type MCoTI-II amino acids 15 and 16 are lysine and lysine, respectively. Fragments of MCoTI-I/II can comprise a fragment in which loop 6 has been deleted, for example, or in which one or more other loops are deleted.

“NBD” or “NEMO Binding Domain” refers the nuclear factor κβ (NF-κβ) essential modulator-binding domain peptide and/or its sequence. “NBD” refers to the full-length sequence of NBD, as represented by SEQ ID NO:4, either with or without a terminal glutamine residue. “NBD or fragments thereof” refers to either the full-length NBD peptide/sequence represented by SEQ ID NO:4, or to pharmaceutically effective fragments of the NBD peptide that can be used to produce the fusion proteins disclosed herein, for example, the NBD fragments represented by SEQ ID NO:22 and SEQ ID NO:23. Fragments of NBD preferably include from about four amino acids to about 10 amino acids of the full-length NBD sequence and can include truncations, amino acid deletions, and/or amino acid modifications of NBD.

“NBD Peptide” refers to a molecule comprising NBD and a CPP (other than MCoTI-I/II or a fragment thereof). NBD Peptides are known to block the activation of the ikB kinase (IKK) kinase complex by preventing the interaction of NEMO with IKKα and β.

“Nucleic acid,” “polynucleotide,” and “nucleotide sequence” are used interchangeably and refer to a polymer of any length of DNA (i.e., cDNA, genomic DNA, plasmids, vectors, viral genomes, isolated DNA, probes, primers, and the like), RNA (i.e., mRNA, antisense RNA), or mixtures of DNA and RNA. Nucleic acids can be single or double-stranded, linear or circular, and/or natural or synthetic polynucleotides. Additionally, nucleic acids can include methylated nucleotides and/or non-naturally occurring nucleotides, such as nucleotide analogs.

“Pharmaceutical” refers to an effect in restoring, correcting or modifying a physiological function of a subject, including the cure, mitigation, treatment or prevention of disease in the subject. A “pharmaceutical composition” and a “medicament” are compositions having a pharmaceutical effect.

“Polypeptide,” “peptide,” and “protein,” may be used interchangeably and refer to polymers of amino acids which compromise four or more amino acids bonded via peptide bonds. A protein can be linear, branched, or cyclic and may comprise naturally occurring amino acids and/or amino acid analogs.

“Subject” refers to an individual treated with a pharmaceutical composition as described herein. The subject is preferably a mammal, and more preferably is a human being.

“Therapeutically effective amount” refers to the amount of a composition that, when administered to a subject, is sufficient to have an effect in restoring, correcting or modifying a physiological function of a subject, including in the treatment of a disease or other medical condition.

“Therapeutic range” refers to the dosage difference between toxic and therapeutic dosages.

“Treatment,” includes both prophylactic treatment and the treatment after a subject experiences a disease or other medical condition, such as inflammation and/or an inflammatory disease or condition. For example, prophylactic treatment of inflammation includes administration of a composition before a subject experiences inflammation and/or an inflammatory disease or condition. “Treating” a disease or condition includes reducing, ameliorating, eliminating, blocking, inhibiting, and the like.

“Comprise” and variations of this term, such as “comprising” and “comprises,” are not intended to exclude other additives, components, integers or steps. The terms “a,” “an,” and “the” and similar referents used herein are to be construed to cover both the singular and the plural unless their usage in context indicates otherwise.

“Consisting essentially of” and variations of the term, shall mean excluding other components or steps of essential significance. Thus, a composition consisting essentially of the recited components would not exclude trace contaminants and pharmaceutically acceptable carriers.

“Consisting of” shall mean excluding more than trace amounts of other components or steps. For example, a polypeptide “consists of” an amino acid sequence when the polypeptide does not contain any other amino acids but the recited amino acid sequence, whereas a polypeptide “consists essentially of” an amino acid sequence when such an amino acid sequence is present together with only a few additional amino acid residues, i.e., from about 1 to about 10 additional residues, more preferably between 1 and 5. A polypeptide “compromises” an amino acid sequence when the amino acid sequence is at least part of the final amino acid sequence of the polypeptide.

### Fusion Proteins

The present invention is directed to a fusion protein compromising a molecule of NBD, or a fragment thereof, fused to MCoTI-I/II or a fragment thereof. NBD is known to block activation of NF-κβ by blocking the activation of the Iκβ complex in the NF-κβ signaling pathway. Blocking activation of Iκβ prevents activation of NF-κβ, thereby preventing it from entering the cytoplasm of the cell and acting as a transcription factor. One such fusion protein is NBD-MCoTI-II, which can be used as a medicament in methods to treat inflammatory diseases and other medical conditions.

NBD

The NBD used in the present fusion molecule is preferably the 11 amino acid sequence as represented by SEQ ID NO:4. However, therapeutically active fragments of this 11 amino acid sequence can also be used to form the present fusion proteins. For example, a 6 amino acid NBD sequence (deletion of the first three amino acids at positions 1-3 and deletion of the last two amino acids at positions 10-11 of NBD), as represented by SEQ ID NO:22, can be fused to MCoTI-I/II or a fragment thereof to form a molecule of the present invention. Alternatively, the 9 amino acid sequence represented by SEQ ID NO:23 (a deletion of amino acids at positions 10-11 of NBD) can be fused to MCoTI-I/II or a fragment thereof.

MCoTI-I/II

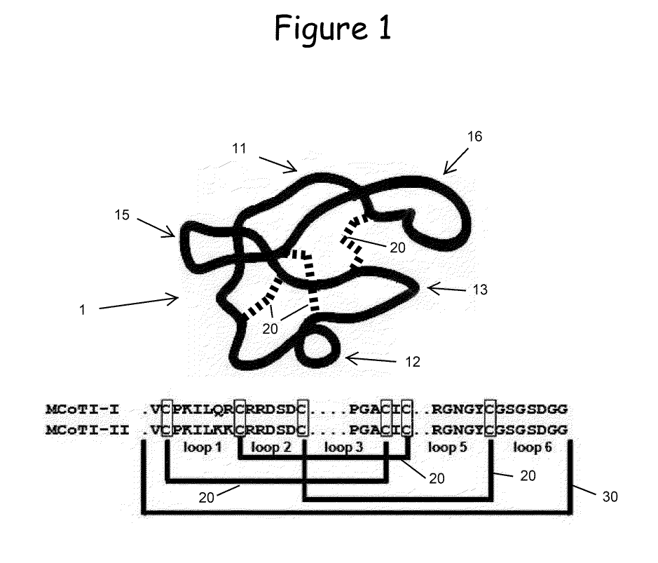

MCoTI-I (SEQ ID NO:24) and MCoTI-II (SEQ ID NO:3) are cystine-knot microproteins (CKMs). In their native forms, these polypeptides (1, shown in FIG. 1) comprise a plurality of loops, including loop 1 (11), loop 2 (12), loop 3 (13), loop 5 (15), and loop 6 (16), and disulfide bonds (20). MCoTI-I and MCoTI-II can be found or produced as cyclic peptides (with a peptide bond shown by reference numeral 30), or can be produced as linear constructs.

CKMs are small peptides, typically consisting of less than 50 amino acids. They are pharmacologically active substances with a defined structure, generally based on intra-molecular disulfide bonds and a small triple stranded β-sheet [(see, e.g., Craik D J (2001) Plant cyclotides: circular, knotted peptide toxins. Toxicon 39:1809-13, the contents of which are herein incorporated by reference in its entirety)]. Their unique structure is responsible for their incredibly high thermal, chemical and enzymatic stability. CKMs can be boiled, incubated at 65° C. for weeks, or even placed in 1N HCl or 1N NaOH without loss of structural and functional integrity. [(see, e.g., Austin J (2009) Biosynthesis and biological screening of a genetically encoded library based on the cyclotide MCoTI-I. Chembiochem 10:2663-70; Fridman J S, et al., (2010) Selective inhibition of JAK1 and JAK2 is efficacious in rodent models of arthritis: preclinical characterization of INCB028050. J Immunol 184:5298-307; Khaja K et al., (2010) Comparison of Functional Protein Transduction Domains Using the NEMO Binding Domain Peptide. Pharmaceuticals 3:110-124; Kimura R H et al., (2009) Engineered knottin peptides: a new class of agents for imaging integrin expression in living subjects. Cancer Res 69:2435-42; and Peterson S et al., (2011) Effects of Portabella mushrooms on collagen-induced arthritis, inflammatory cytokines, and body composition in dilute brown non-agouti (DBA1) mice. Functional Foods in Health and Disease 1:279-296)]. CKMs include the intrathecally administered analgesic drug ziconotide (see, e.g., Doggrell S A (2004) Intrathecal ziconotide for refractory pain. Expert Opin Investig Drugs 13:875-7).

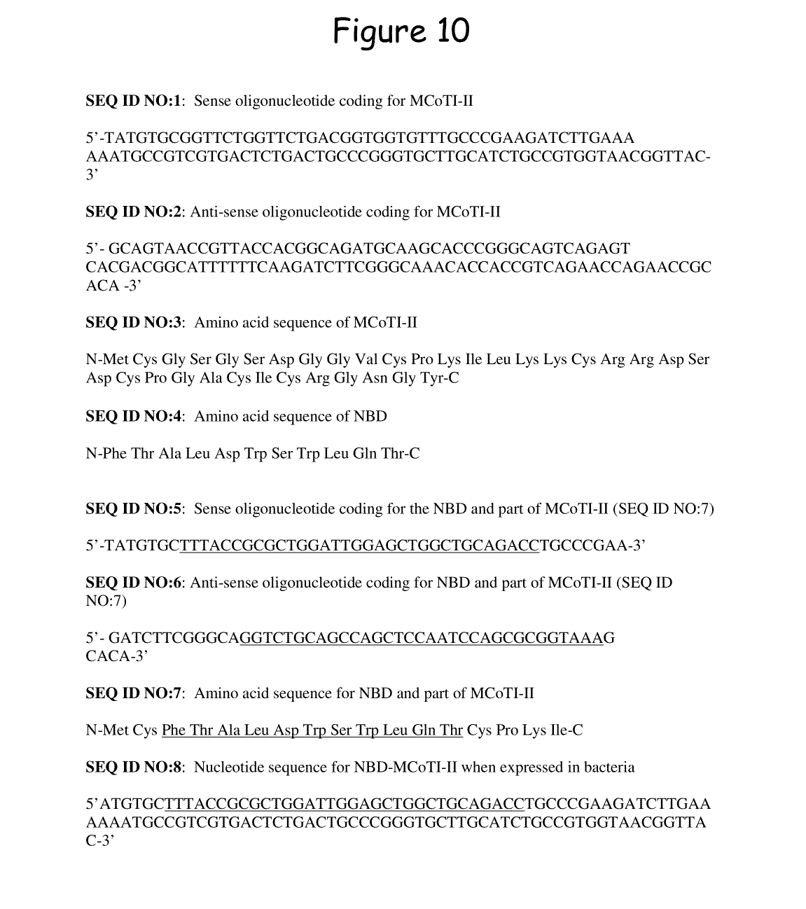

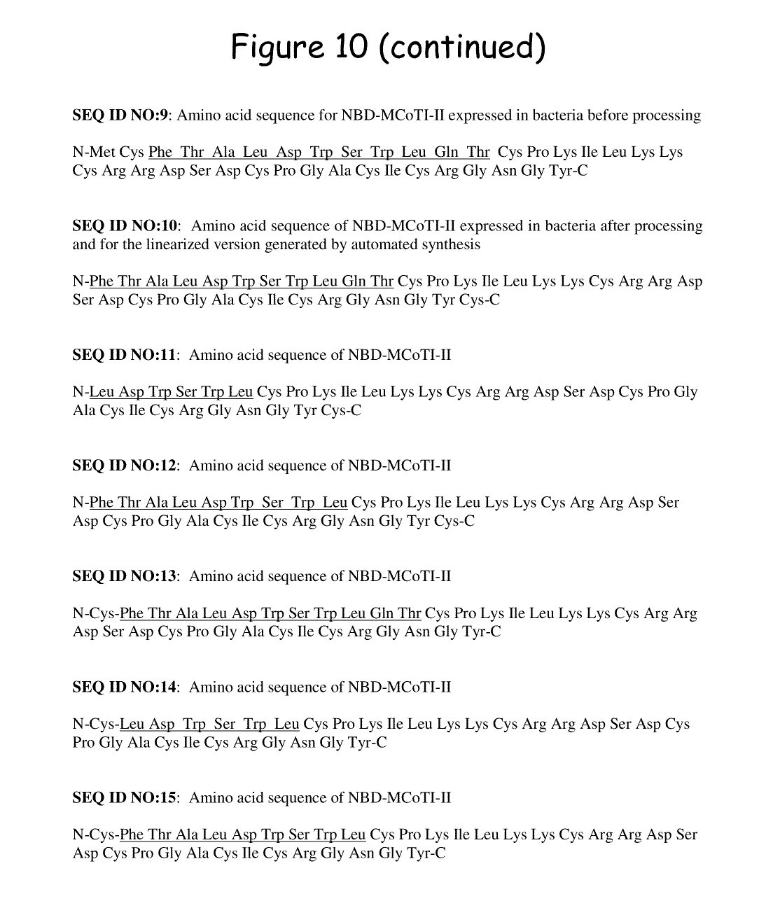

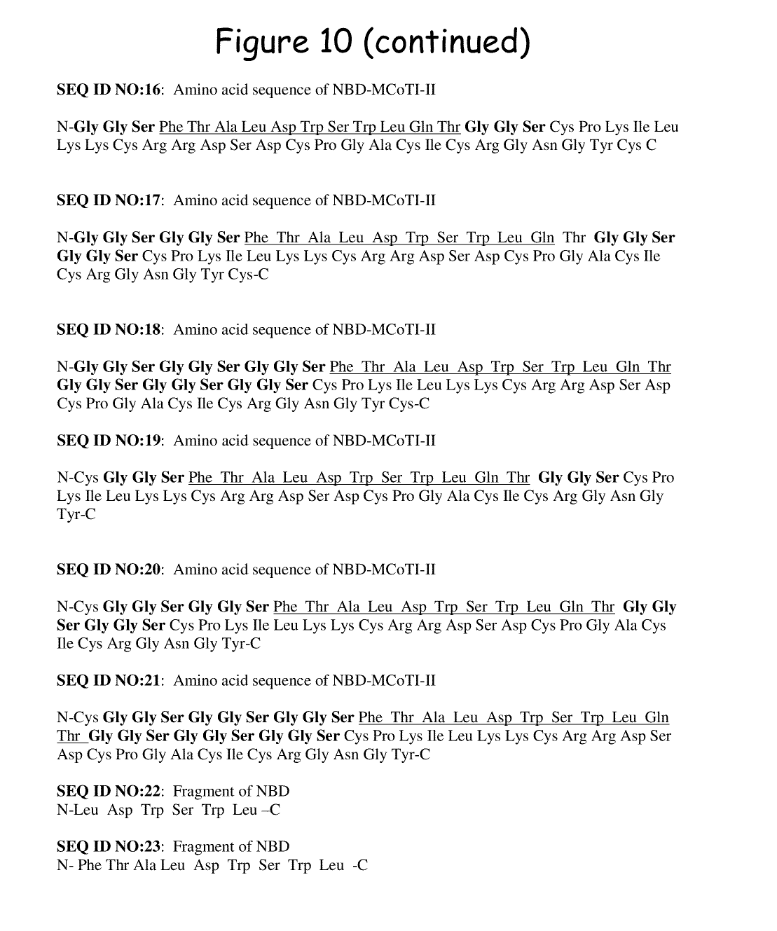

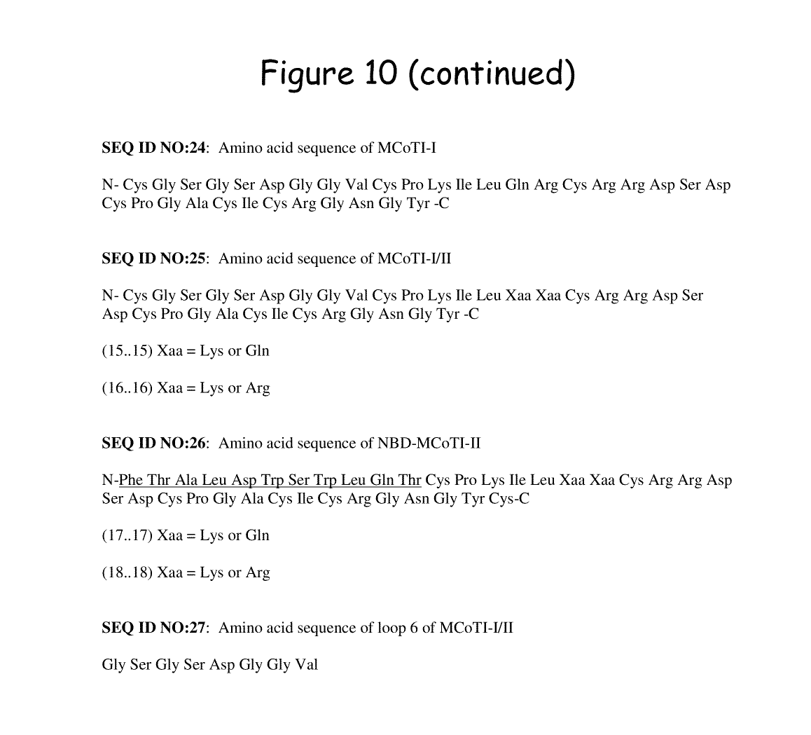

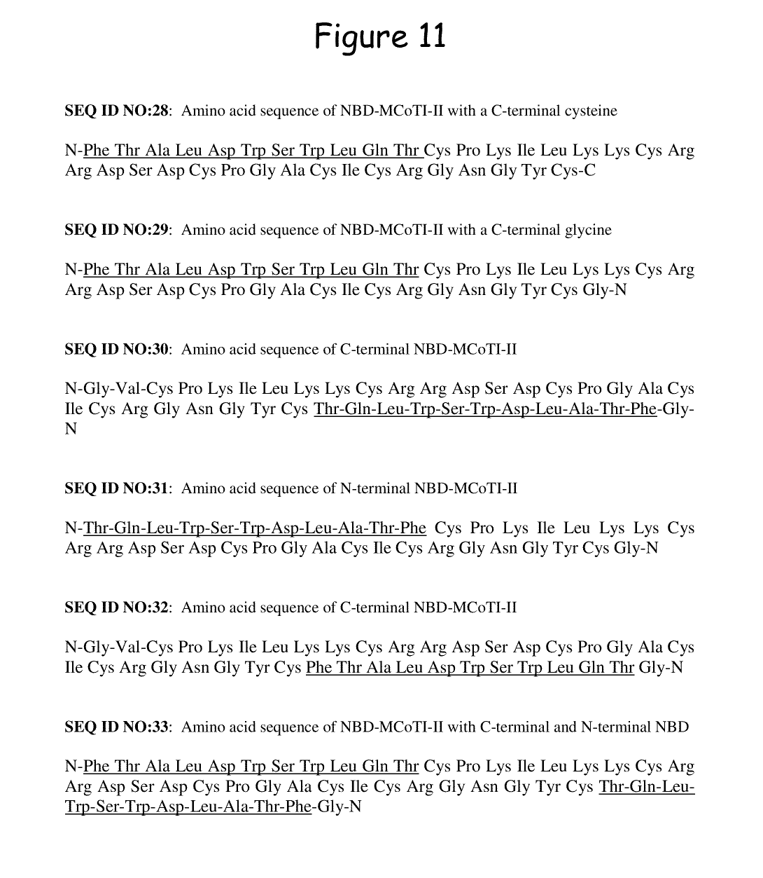

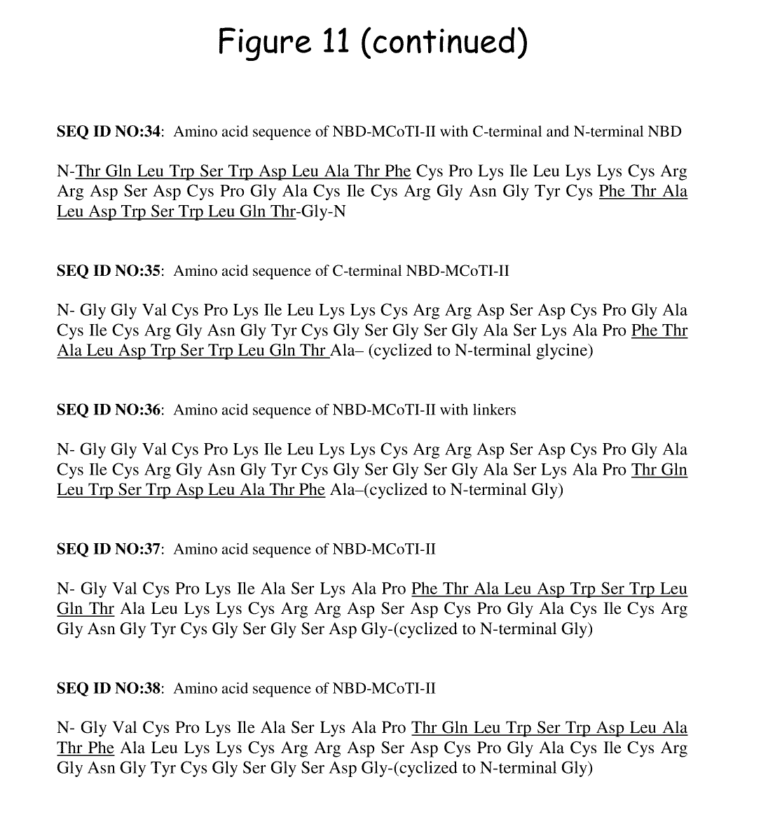

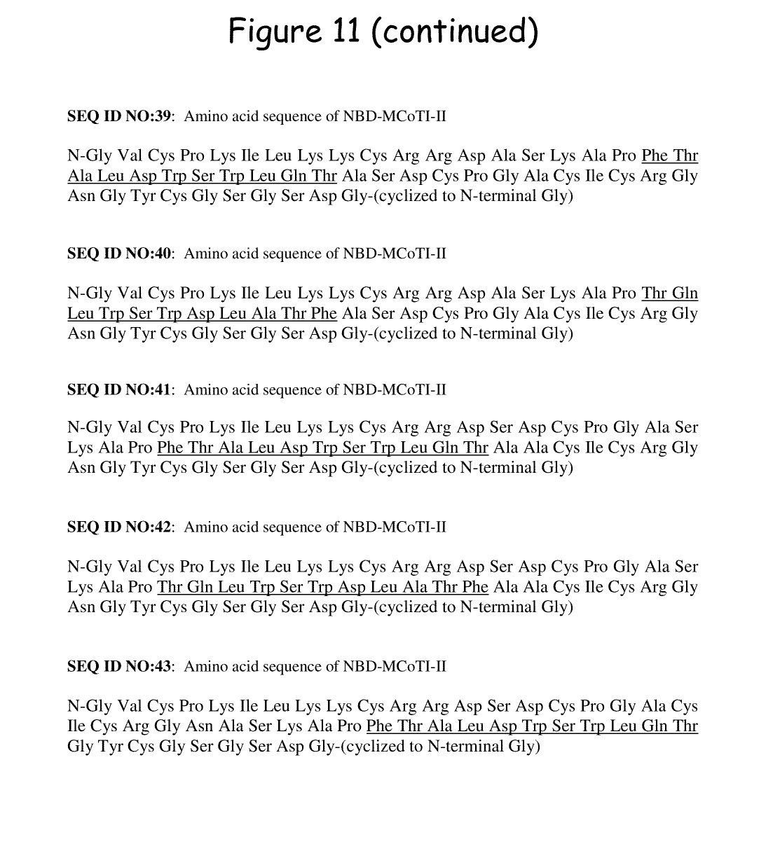

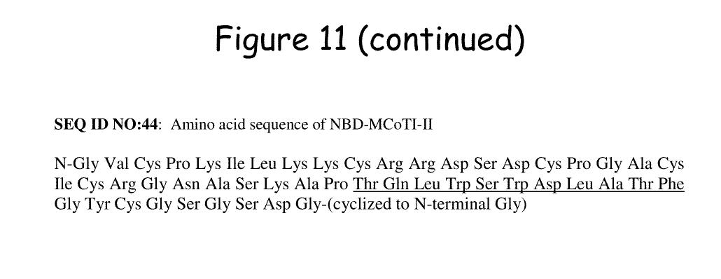

MCoTI-I or MCoTI-II or fragments thereof form the backbone of the fusion proteins disclosed herein. The sequence of MCoTI-I is shown by SEQ ID NO: 25 (FIG. 13), while that of MCoTI-II is shown by SEQ ID NO:3 (FIG. 10). MCoTI-I and MCoTI-II are members of the trypsin inhibitor CKM subfamily.

NBD-MCoTI-II

NBD-MCoTI-I/II as disclosed herein is formed by the fusion of (1) NBD (represented by SEQ ID NO:4), or a fragment thereof, and (2) MCoTI-I/II (represented by SEQ ID NO:25), or a fragment thereof. The fusion can be direct, i.e. without any amino acid residues in between the peptide units, or can be via one or more linkers. NBD-MCoTI-I/II can be produced and used either in cyclic or linear form.

In one example, MCoTI-II in linear form can be used to form the present NBD-MCoTI-II fusion protein, and can be represented by SEQ ID NO:10. Preferably, the fusion protein is formed by replacing loop 6 of MCoTI-I/II (as shown in FIG. 1) with NBD or a fragment thereof. Loop 6 of MCoTI-II is represented by SEQ ID NO: 27. As such, in the final fusion protein, the foregoing amino acid sequence is preferably not present.

In other embodiments, the inventive fusion protein can be circular and/or can be both linear and circular. In yet other embodiments, the NBD, or a fragment thereof, can be inserted into other locations in the MCoTI-II protein sequence. For example, the inventive fusion proteins disclosed herein can be formed by replacing loop 6 and at least a portion of one or more other loops of MCoTI-II and/or the inventive fusion proteins disclosed herein can be formed by replacing at least a portion of loop 6 and/or the inventive fusion proteins disclosed herein can be formed by replacing at least a portion of a loop other than loop 6 of MCoTI-II.

In one embodiment, the NBD-MCoTI-II can be represented by SEQ ID NO:11. In this fusion protein, the NBD has a three amino acid deletion at positions 1-3, and a two amino acid deletion at positions 10 and 11. This NBD fragment is represented by SEQ ID NO:22. This NBD fragment is preferably located at the N-terminus of the fusion protein. In other embodiments, the NBD fragment described above may not be located at the N-terminus of the fusion protein, as represented by, for example, SEQ ID NO:14. For example, the NBD fragment can follow one or more linkers that are immediately at the N-terminus of the fusion protein.

NBD-MCoTI-II can alternatively be represented by SEQ ID NO:12. In this fusion protein, the NBD has a two amino acid deletion at positions 10 and 11. This NBD fragment is represented by SEQ ID NO:23. The NBD fragment is located at the N-terminus of the fusion protein. In other embodiments, the NBD fragment described above may not be located at the N-terminus of the fusion protein, as represented by, for example, SEQ ID NO:15. For example, this NBD fragment can follow one or more linkers that are immediately at the N-terminus of the fusion protein.

In another embodiment, NBD-MCoTI-II can be represented by SEQ ID NO:13. In this fusion protein, the NBD not immediately at the N-terminus of the MCoTI-I/II backbone. NBD-MCoTI-II can also be represented by SEQ ID NOS:16-21, where the NBD (or a fragment thereof) is flanked by one or more linkers. The linkers can be located at or near the N-terminus, and/or at or near the C-terminus, of the fusion protein. Linkers can be used to increase the flexibility of NBD (or a fragment thereof) within the framework of MCoTI-I/II. Preferably, the linker is Gly Gly Ser. Linkers are well known. Any type and number of linkers can be used. For example, more than 3 amino acid linkers can be used, and/or longer linkers can be used. In other embodiments, linkers can be omitted.

In alternative embodiments, the NBD-MCoTI-I/II peptides can contain subsets of the NBD sequence (SEQ ID NO:11, SEQ ID NO:12), or in which the NBD sequence is not immediately at the N-terminus (SEQ ID NO:13, SEQ ID NO:14, SEQ ID NO:15). The NBD sequence can also alternatively contain one (SEQ ID NO:16, SEQ ID NO:19), two (SEQ ID NO:17, SEQ ID NO:20) or three (SEQ ID NO:18, SEQ ID NO:21) linkers (Gly Gly Ser) at the N- and C-termini of NBD to increase the flexibility of NBD within the framework of the MCoTI-II backbone.

MCoTI-I and MCoTI-II differ from each other by only two amino acid residues. It has been found that grafting the NBD sequence into MCoTI-I (in all the variations described herein) results in NBD-MCoTI-I peptides with similar biological activity.

Linear MCoTI-I/II

In one embodiment, the present invention can comprise the use of a linear MCoTI-I/II molecule in which the NBD peptide is fused (or otherwise covalently linked) to the N terminus of a MCoTI-I/II molecule. In this linear version of NBD-MCoTI-II the sequence corresponding to Loop 6 in the cyclized version is preferably replaced by the NBD sequence on the N-terminus (SEQ ID NO:29). The NBD sequence protrudes out from the MCoTI-II backbone and has sufficient flexibility to achieve a bioactive conformation necessary for inhibiting the IKKβ-NEMO interaction.

The NBD sequence grafted in the reverse orientation onto the C-terminal region of MCoTI-II (SEQ ID NO:30) results in a peptide conformationally similar to the N-terminal NBD-MCoTI-II peptide (SEQ ID NO:29). Furthermore, grafting the NBD sequence in the alternative orientation for both the linear N- and C-terminal versions results in bioactive peptides (SEQ ID NO:31 and SEQ ID NO:32).

Biologically, the interaction of IKKβ with NEMO consists of a two IKKβ-proteins interacting with two NEMO proteins in a dimer of dimers configuration. Similarly, grafting the NBD sequence simultaneously into the N and C termini of linear MCoTI-II allows the NBD-MCoTI-II peptide to interact with NEMO in a similar manner as IKKβ-NEMO resulting in NBD-MCoTI-II having improved potency (SEQ ID NO:33 and SEQ ID NO:34). In the molecules of SEQ ID NO:33 and SEQ ID NO:34, the NBD molecule on one terminus is synthesized in reverse order and comprises the sequence Thr Gln Leu Trp Ser Trp Asp Leu Ala Thr Phe (SEQ ID NO:45). The NBD sequences are preferably in the same orientation, but can also be in reverse orientations.

Cyclized Versions of NBD-MCoTI-II

Grafting the NBD sequence into various loops of cyclized MCoTI-II preferably includes the use of a linker so as not to limit the flexibility of the grafted peptide such that a conformationally functional folded state can be achieved. The inclusion of a linker also allows flexibility in the backbone so that MCoTI-II can undergo proper folding/disulfide bond formation/cyclization. For example, NBD sequence with linkers inserted in the middle of Loop 6 in both orientations (SEQ ID NO:35 and SEQ ID NO:36) results in inhibition of IKβ-NEMO interactions. Similarly, the NBD sequence grafted into Loops 1, 2, 3 and 5 in both orientations (SEQ ID NO:37, SEQ ID NO:38, SEQ ID NO:39, SEQ ID NO:40, SEQ ID NO:41, SEQ ID NO:42, SEQ ID NO:43, SEQ ID NO:44) produces a peptide with the same intended biological activity.

### Methods of Manufacturing

The present fusion proteins can be manufactured by methods known to the art. For example, automated polypeptide synthesis can be performed to produce the present proteins, such as Fmoc-based solid phase peptide synthesis. Typically peptides are synthesized as trifluoroacetic acid (TFA) salts. The TFA salt may not be compatible with in vivo administration, and usually the counter ion is changed to acetate. We have therefore synthesized linear N-terminal MCoTI-II (SEQ ID NO:3) as both the TFA and acetate salts. The difference in counter ion had no effect on the in vitro and in vivo biological activity of the linear NBD-MCoTI-II, however.

We have synthesized different linear N-terminal NBD-MCoTI-II peptides in which the C-terminal residue was a cysteine (SEQ ID NO:28) and a glycine (SEQ ID NO:29). We also changed the carboxyl group to a carboxamide to improve on the efficiency of synthesis (SEQ ID NO:29). Having a carboxamide attached to the solid support is much more stable than ester attachments to anhydrous acids so that premature loss of peptide due to acid hydrolysis is not a problem during manufacturing. Having different residues or a carboxamide on the C-terminus did not alter the in vitro and in vivo biological activity of N-terminal NBD-MCoTI-II.

Alternatively, the present fusion proteins can be produced by recombinant methods, such as by using bacterial, yeast, insect, or mammalian cells. A variety of cell lines can be used to produce the present proteins recombinantly, such as HEK cells, CHO cells, HeLa cells, and others known to the art. Suitable polynucleotides and vectors are selected when producing the present fusion proteins recombinantly. For example, the isolated polynucleic acid molecule represented by SEQ ID NO:8 can be used for bacterial synthesis. Preferably, the polynucleic acid can be DNA or RNA. Preferably, vectors include all of the regulatory elements necessary for efficient transfection as well as efficient expression of proteins. Such vectors are well known in the art and any suitable vector can be selected for this purpose. Transfection and cultivation of recombinant cells is likewise well known in the art. Preferably, such a recombinant cell as well as any descendant cell thereof includes a vector comprising a nucleotide sequence coding for the preset fusion proteins. Such cell lines can express the fusion proteins described herein (e.g., the NBD-MCoTI-II) continuously or upon induction/activation depending on the vector.

Polynucleotides coding for the present fusion proteins can, in an alternative embodiment be directly administered to a subject in order to deliver the fusion proteins to the subject. Such delivery would be by way of gene therapy vectors and protocols known to the art.

Following manufacture of the present fusion proteins, such proteins can be formulated as a pharmaceutical composition compromising the fusion proteins described herein, i.e., compromising NBD-MCoTI-I/II, and pharmaceutically acceptable excipients. The pharmaceutical composition can include, for example, emulsifiers, buffers, inert/inactive ingredients, sugars, salts, and the like. Preferably, the pharmaceutical composition is administered orally. The pharmaceutical composition, however, may be suitable for administration in any manner, such as, but not limited to, injection, orally, transdermally, and/or topically. For example, if provided in an oral form, the pharmaceutical composition can be formulated to have the following properties: enzymatic resistance, stability in gastric juices and GI membrane permeability.

### Treatments

NBD-MCoTI-I/II can be used as a medicament in methods to treat a number of different medical conditions involving the activation of NF-κβ, in particular inflammatory conditions or diseases, by inhibiting TNFα-mediated activation of the NF-kB pathway. The inventive proteins, polynucleic acids, and/or vectors described herein, can be used as medicaments to treat inflammatory diseases or conditions. In this regard, the present invention is directed to methods for the treatment of inflammatory diseases or conditions, wherein the NBD-MCoTI-II, isolated polynucleic acid molecule and/or vector described herein, and/or a pharmaceutical composition according to the invention, is administered to a patient. The inventive fusion proteins, polynucleic acids, and/or vectors can be administered alone or in addition to other medicaments that treat inflammatory diseases or conditions and/or other diseases and conditions. For example, the inventive fusion proteins, polynucleic acids, and/or vectors can be administered as part of a treatment regimen with other known medicaments. In other embodiments, the inventive proteins, polynucleic acids, and/or vectors described herein can be used as medicaments to treat any other disease or condition that appears to improve upon the administration of the medicament.

The inventive proteins, polynucleic acids, and/or vectors described herein can be used to inhibit and/or block the activation of the NF-κβ pathway. Inflammatory mediators such as TNF-α, IL-6, and IL-1 are important in the pathogenesis of inflammatory diseases and are regulated by the activation of NF-κB, so blocking this molecule can treat such conditions. NBD-MCoTI-I/II has been discovered to be 10⁶-fold more potent than known cell-permeable NBD peptides. This improvement in potency has translated into a reduction in dosage required to ameliorate inflammatory injury in vivo (as shown, for example, in the Carrageenan Paw Edema (CPE) mouse model). Therefore, NBD-MCoTI-I/II is a potent, orally bioavailable agent to treat inflammatory diseases.

The inflammatory diseases and/or conditions that can be treated using the methods described herein, include, but are not limited to, chronic or acute diseases or conditions such as Alzheimer's, ankylosing spondylitis, arthritis (i.e., osteoarthritis, rheumatoid arthritis, psoriatic arthritis), cancer, edema, swelling, asthma, atherosclerosis, Crohn's disease, colitis, dermatitis, diverticulitis, fibromyalgia, hepatitis, irritable bowel syndrome, systemic lupus erythematous, nephritis, Parkinson's disease, ulcerative colitis, acid reflux/heartburn, acne, asthma, atherosclerosis, bronchitis, cancer, carditis, celiac disease, chronic pan, cirrhosis, dementia, diabetes, dry eyes, edema, emphysema, eczema, gastroenteritis, gingivitis, heart disease, high blood pressure, insulin resistance, interstitial cystitis, joint pain/arthritis/rheumatoid arthritis, metabolic syndrome (syndrome X), myositis, nephritis, obesity, osteopenia, osteoporosis, periodontal disease, polychondritis, psoriasis, sinusitis, spastic colon, Sjogren's syndrome, systemic candidiasis, tendonitis, UTIs, vaginitis, respiratory allergic diseases, hypersensitivity lung diseases, anaphylaxis or hypersensitivity responses, drug allergies, insect sting allergies, inflammatory bowel diseases, autoimmune diseases such as, but not limited to, autoimmune hematological disorders, autoimmune thyroiditis, Behcet's disease, Grave's disease, graft rejection, graft vs. host disease, and the like. Additional conditions and diseases include ischemia reperfusion injury, cytokine induced toxicity, and the like. Preferably, conditions such as arthritis and swelling are treated.

The inventive fusion proteins, polynucleic acids, and/or vectors disclosed herein can be used to prevent certain diseases or conditions, such as to prevent certain inflammatory diseases or conditions. For example, the inventive fusion proteins, polynucleic acids, and/or vectors disclosed herein can be used to prevent arthritic joint damage.

Other conditions in which NF-κB plays a role in pathology can also be treated with the present invention, such as musculoskeletal diseases such as muscular dystrophy and cachexia, in which the activation of nuclear factor κB (NF-κB) contributes to muscle degeneration that results from dystrophin deficiency in human Duchenne muscular dystrophy (DMD). Also included are cardiac conditions such as cardiac hypertrophy, ischemia, and reperfusion injury; spinal cord injury; sepsis; and cancer. For example, in large B-cell lymphoma, constitutive NF-κB activity can be suppressed by NBD-MCoTI-I/II.

### EXAMPLES

### Synthesis of NBD-MCoTI-II

The NBD sequence (SEQ ID NO:4) was grafted into MCoTI-II (SEQ ID NO:3) by replacing the loop 6, as shown in FIG. 1. Synthesis was accomplished in two ways: 1) expression in bacteria [(see, e.g., Austin J et al., (2009) Biosynthesis and biological screening of a genetically encoded library based on the cyclotide MCoTI-I. Chembiochem 10:2663-70 and Schmoldt H U et al., (2005) A fusion protein system for the recombinant production of short disulfide bond rich cystine knot peptides using barnase as a purification handle. Protein Expr Purif 39:82-9); the contents of each of which are herein incorporated by reference in their entireties] and 2) synthetically on an automated peptide synthesizer [(see, e.g., Avrutina O et al., (2004) Fmoc-Assisted Synthesis of a 29-Residue Cystine-Knot Trypsin Inhibitor Containing a Guaninyl Amino Acid at the P1-Position. European Journal of Organic Chemistry 2004:4931-4935 and Camarero J A et al. (2004) Fmoc-based synthesis of peptide alpha-thioesters using an aryl hydrazine support. J Org Chem 69:4145-51)].

NBD-MCoTI-II was synthesized using both bacterial and automated synthetic approaches. Bacterial synthesis resulted in circular, native-folded NBD-MCoTI-II. For the automated synthesis we generated a linear version of NBD-MCoTI-II. Both approaches are described in more detail below.

### Bacterial Synthesis of NBD-MCoTI-II. Generation of NBD-MCoTI-II Construct

A gene coding for the NBD-MCoTI-II, generated using complimentary sense and anti-sense oligonucleotides, was subcloned into an appropriate plasmid for protein expression in bacteria. When expressed in bacteria, NBD-MCoTI-II was purified in its native folded conformation. However, NBD-MCoTI-II had to be purified away from bacterial proteins using multiple columns/HPLC. Wild-type MCoTI-II-intein-chitin binding domain (CBD) fusion protein was generated as follows. Sense (SEQ ID NO:1) and anti-sense (SEQ ID NO:2) oligonucleotides coding for the protein sequence (SEQ ID NO:3) of MCoTI-II were annealed to generate a double-stranded cassette. 5′ Nde I and 3′ Sap I sites were included for subcloning the cassette into the vector pTXB1 (which contains the intein-CBD coding regions). A Bgl II restriction site was also included after loop 6 to allow for subcloning in cDNA sequences coding for the NEMO Binding Domain sequence (NBD) (SEQ ID NO:4). The cDNA sequence for MCoTI-II was subcloned into pTXB1 such that it was immediately upstream and in frame with the cDNA region coding for the intein-CBD coding region.

A NBD-MCoTI-II cassette was generated by annealing sense (SEQ ID NO:5) and anti-sense (SEQ ID NO:6) oligonucleotides coding for the NBD sequence and part of the MCoTI-II peptide (SEQ ID NO:7). 5′ Nde I and 3′ Bgl II sites were included for subcloning the NBD coding sequence into wildtype MCoTI-II-intein-CBD:pTXB1. Subcloning the NBD coding region into the Nde I/Bgl II sites of MCoTI-II:pTXB1 replaced the wildtype loop 6 coding region with the NBD coding region resulting in the construct NBD-MCoTI-II:pTXB1. Nucleotide and protein sequences for expression of NBD-MCoTI-II in bacteria are given in SEQ ID NO:8 and SEQ ID NO:9, respectively.

### Expression and Purification of NBD-MCoTI-II

Bacterial expression of wildtype and NBD-MCoTI-II was carried out in Origami E. coli (EMD Chemicals, Inc.). E. coli transfected with either wildtype MCoTI-II:pTXB1 or NBD-MCoTI-II:pTXB1 were grown at 37° C. in LB medium in the presence of ampicillin until log phase growth was reached. After reaching log phase growth, the cells were acclimated to 30° C., IPTG added (at a final concentration of 0.3 μM) and grown at 30° C. for two hours. Cells were harvested by centrifugation and stored at −80° C.

E. coli expressing wildtype or NBD-MCoTI-II were lysed in Bugbuster solution (EMD Chemicals, Inc.) containing protease inhibitors and benzonase at room temperature for 20 minutes. Cleared lysate, obtained by centrifugation (10,000×g, 4° C., one hour), was added to chitin beads (available from NEB, Inc.) (washed with 30 volumes of HEPES buffer: 20 mM HEPES, pH 8.0, 500 mM NaCl and 500 MgCl₂) and incubated at 4° C. with mixing for one hour. A column was poured and washed with 50 volumes of HEPES buffer with 0.1% Triton-X-100 followed by 50 volumes of HEPES buffer. The column was treated overnight with 30 mM DTT in HEPES buffer resulting in the elution of cyclized and folded wildtype or NBD-MCoTI-II. The elution was lyophilized, resuspended in sterile water and wildtype or NBD-MCoTI-II was purified by reversed phase HPLC on a C18 column at a flow-rate of 1.0 ml/min using a linear gradient of 0-60% solvent B (90% ACN/0.1% TFA) in solvent A (0.1% aqueous TFA). The active peak was collected, ACN removed by roto-evaporation, and lyophilized. The final NBD-MCoTI-II protein sequence after processing can be represented by SEQ ID NO:10 (though it is to be understood that the product of the foregoing process is a circular protein, and the designation of N- and C-termini in SEQ ID NO:10 is arbitrary).

### Automated Synthesis of NBD-MCoTI-II

NBD-MCoTI-II was chemically synthesized by Fmoc-based solid-phase peptide synthesis followed by optional head to tail cyclization. When automated synthesis is used the peptide may have to be folded to the native conformation [(see, e.g., Aboye T L et al., (2011) Interlocking disulfides in circular proteins: toward efficient oxidative folding of cyclotides. Antioxid Redox Signal 14:77-86; Cemazar M et al., (2006) Knots in rings. The circular knotted protein Momordica cochinchinensis trypsin inhibitor-II folds via a stable two-disulfide intermediate. J Biol Chem 281:8224-32; and Cemazar M et al., (2008) The structure of a two-disulfide intermediate assists in elucidating the oxidative folding pathway of a cyclic cystine knot protein. Structure 16:842-510); the contents of which are herein incorporated by reference in their entireties]. To accomplish this, NBD-MCoTI-II was placed in buffers containing reduced and oxidized glutathione. Correct folding was determined by 1H-1H TOCSY NMR (see, e.g., Austin J et al., (2009) Biosynthesis and biological screening of a genetically encoded library based on the cyclotide MCoTI-I. Chembiochem 10:2663-70, the contents of which are herein incorporated by reference in its entirety).

We designed a linear version of NBD-MCoTI-II (SEQ ID NO:10) where loop 6 was replaced with the NBD sequence (SEQ ID NO:4). We utilized automated chemical synthesis as described above to generate linear NBD-MCoTI-II.

### Inhibition of NF-κB Pathway by NBD-MCoTI-II

A cell-permeant NBD Peptide has been demonstrated in in vitro binding and cell-based assays as well as in vivo rodent models to inhibit NF-κB signaling and inflammatory responses [(see, e.g., Dai S et al., (2004) The IkappaB kinase (IKK) inhibitor, NEMO-binding domain peptide, blocks osteoclastogenesis and bone erosion in inflammatory arthritis. J Biol Chem 279:37219-22; Darwech I et al., (2009) Impediment of NEMO oligomerization inhibits osteoclastogenesis and osteolysis. J Cell Biochem 108:1337-45; di Meglio P et al., (2005) Amelioration of acute inflammation by systemic administration of a cell-permeable peptide inhibitor of NF-kappaB activation. Arthritis Rheum 52:951-8; Khaja K et al., (2010) Comparison of Functional Protein Transduction Domains Using the NEMO Binding Domain Peptide. Pharmaceuticals 3:110-124; May M J, Marienfeld R B, and Ghosh S (2002) Characterization of the Ikappa B-kinase NEMO binding domain. J Biol Chem 277:45992-6000; Peterson J M et al., (2011) Peptide-based inhibition of NF-kappaB rescues diaphragm muscle contractile dysfunction in a murine model of Duchenne muscular dystrophy. Mol Med; Tilstra J et al., (2007) Protein transduction: identification, characterization and optimization. Biochem Soc Trans 35:811-5), the contents of each of which are herein incorporated by reference in their entireties].

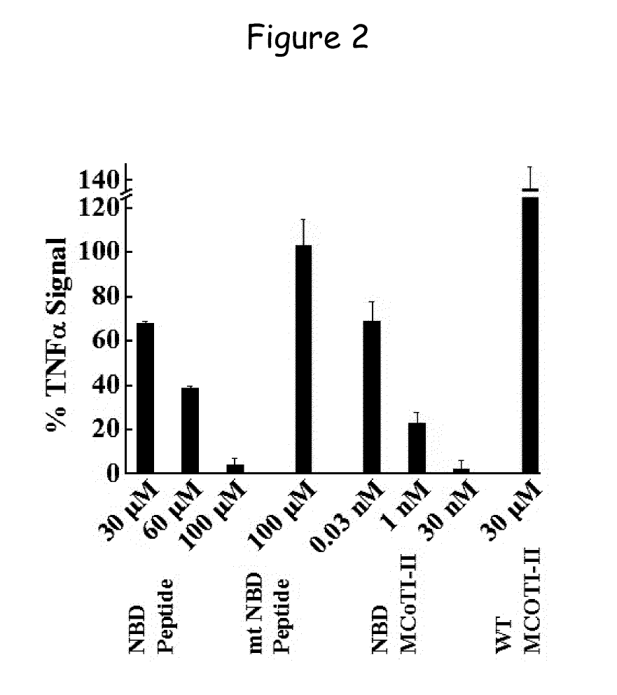

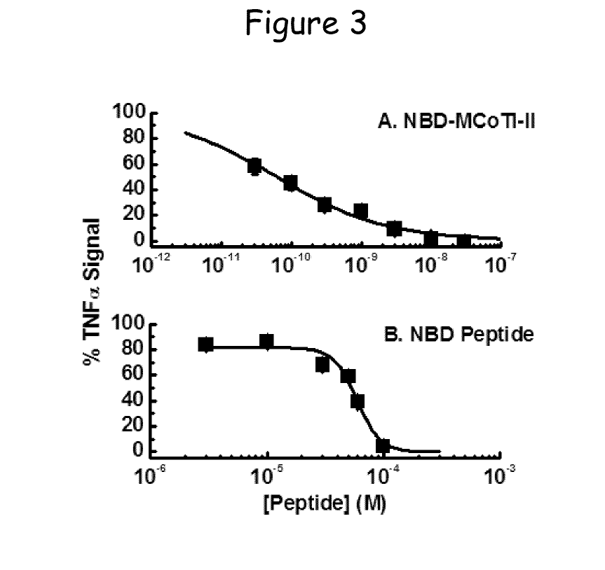

To demonstrate that a grafted NBD sequence into MCoTI-II still exhibited the ability to inhibit NF-κB signaling we utilized a cell-based assay. HEK293 cells were transfected with the plasmid pNiFty-SEAP which encodes for a secreted alkaline phosphatase (SEAP) whose expression is under control of an ELAM-1 composite promoter containing five NF-κB sites. Treatment of cells with TNFα causes activation of the IkB kinase complex and, in turn, NF-κB, resulting in SEAP expression. We verified that pNiFty-SEAP transfected HEK293 cells treated with the NBD Peptide (FIG. 2; FIG. 3, Panel B) blocked the expression of TNFα-mediated SEAP expression in a concentration-dependent manner with an IC₅₀ of ˜60 μM. As a control, a mutant, inactive form of NBD Peptide did not block TNFα-mediated SEAP expression (FIG. 2). Treatment of cells with circularized NBD-MCoTI-II generated in bacteria (data not shown) or linear NBD-MCoTI-II generated by automated synthesis (FIG. 2; FIG. 3 Panel A) blocked TNFα-mediated SEAP expression, but not WT MCoTI-II (FIG. 2). This result shows that the NBD sequence grafted into the backbone of MCoTI-II retains the ability to enter the cell and inhibit NF-κB signaling in the cytoplasm. Most importantly, linear NBD-MCoTI-II blocked TNFα-mediated SEAP expression with an IC₅₀ of 60 pM, 10⁶-fold more potent than that of the NBD peptide. Finally, WT and NBD-MCoTI-II caused no overt cellular toxicity at concentrations up to 1 μM.

NBD-MCoTI-II Reduces in vivo Inflammatory Response

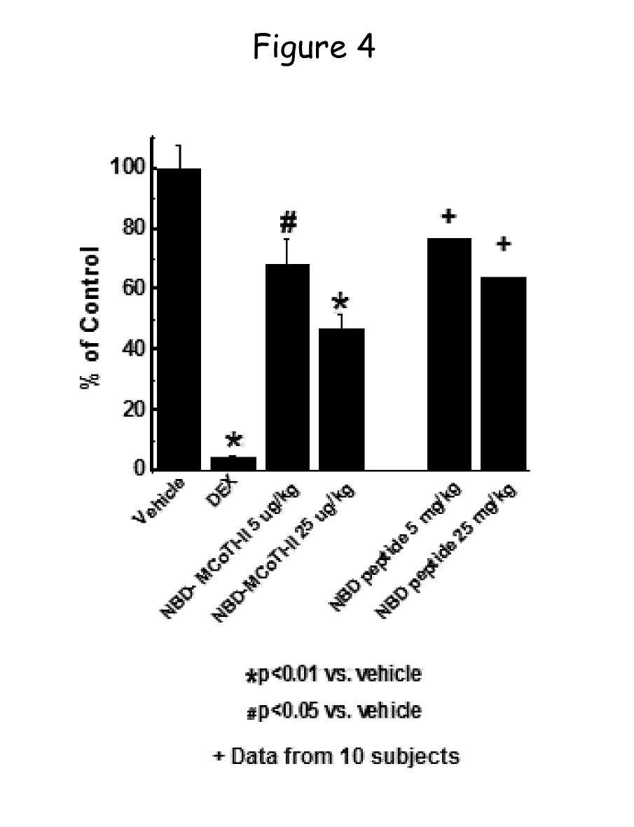

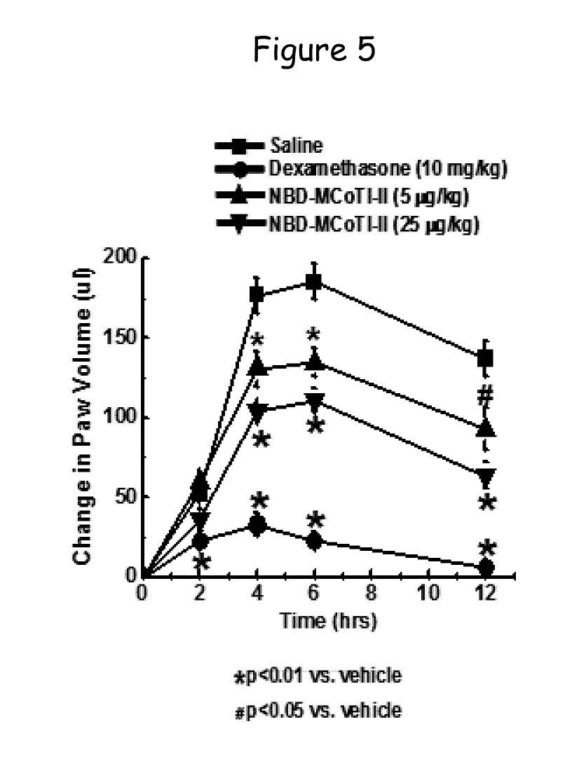

Anti-inflammatory activity of linear NBD-MCoTI-II was tested at two doses (5 and 25 μg/kg) in the mouse CPE model. Mice were administered (i.p.) either NBD-MCoTI-II, dexamethasone (10 mg/kg) or saline one hour prior to inducing edema by carrageenan injection into the subplantar region of the left hind foot. Swelling was then measured by water displacement method at 2, 4, 6 and 12 hours post-carrageenan injection. Linear NBD-MCoTI-II significantly reduced swelling at 4, 6 and 12 hours (FIG. 4). At 12 hours, 5 and 25 μg/kg NBD MCoTI-II reduced swelling 30 and 50%, respectively (FIG. 5). As a comparison, NBD-MCoTI-II at 5 μg/kg had a similar effect as the NBD Peptide at 25 mg/kg (FIG. 4., di Meglio P et al., (2005) Amelioration of acute inflammation by systemic administration of a cell-permeable peptide inhibitor of NF-κβ activation. Arthritis Rheum 52:951-8, the contents of which are herein incorporated by reference in its entirety), indicating that the in vivo potency of linear NBD-MCoTI-II is 5000-fold greater than that observed for the NBD Peptide. (FIG. 5).

### NBD-MCoTI-II Reduces Inflammatory Response in a Therapeutic Mouse CIA Model

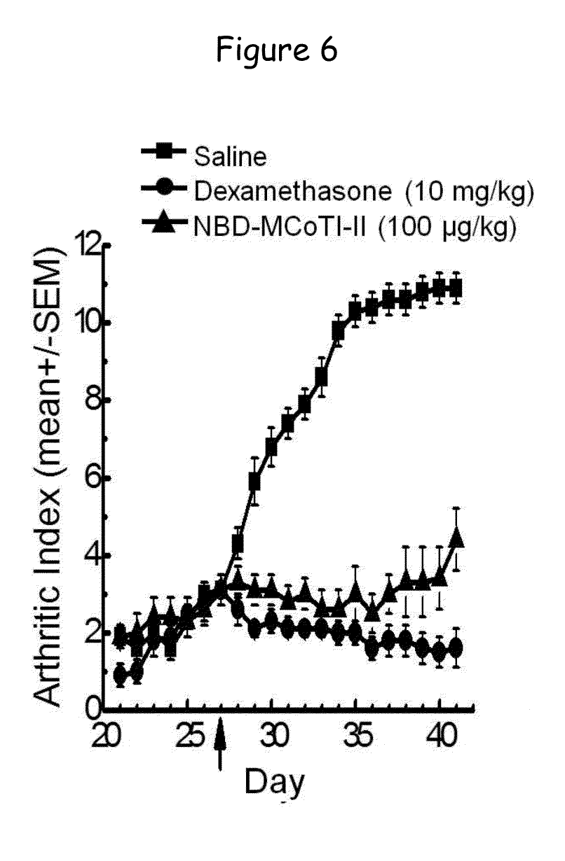

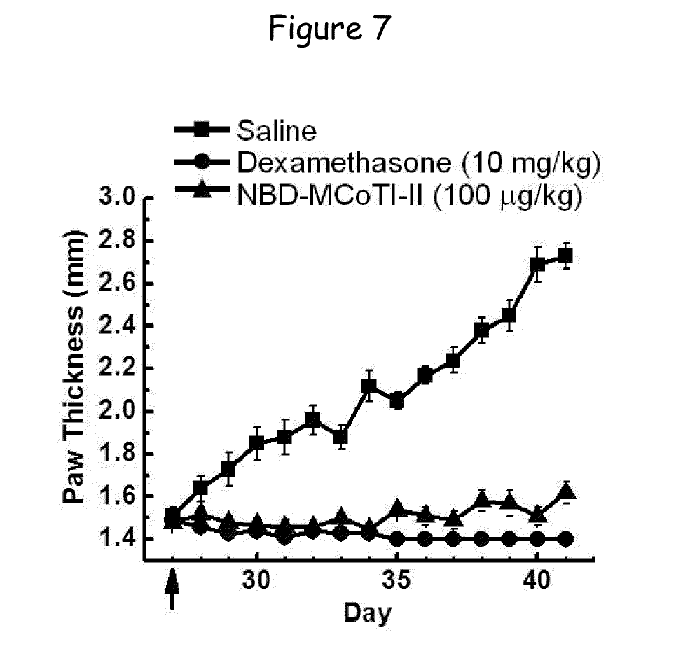

We next tested the efficacy of linear NBD-MCoTI-II in the mouse CIA model in therapeutic mode, a standard model for evaluating anti-arthritic activity. Mice were injected with collagen type II. CIA was induced in male DBA/1J mice by tail injection of Type II collagen (100 μg) mixed with complete Freund's adjuvant. On Day 21, a booster injection (i.p.) of collagen (100 μg)/incomplete Freund's adjuvant was administered. The severity of arthritis (arthritic index (AI)) was scored in each paw as follows: 0-no evidence of erythema and swelling, 1-erythema and mild swelling confined to the mid-foot (tarsals) or ankle joint, 2-erythema and mild swelling extending from the ankle to the mid-foot, 3-erythema and moderate swelling extending from the ankle to the metatarsal joints and 4-erythema and severe swelling encompass the ankle, foot and digits. AI scores were measured in each paw and summed (maximum score of 16 per mouse). After the AI score reached an average of 3.1 on Day 27, mice were separated into three treatment groups and administered (i.p.) daily: saline, 100 μg/kg NBD-MCoTI-II and 3 mg/kg dexamethasone for 14 days. AI scores, body weights, paw thickness and any signs of distress were monitored daily. By Day 29 NBD-MCoTI-II (100 μg/kg) immediately and significantly (p<0.01) halted the progression of arthritis similar to that of the positive control dexamethasone (FIG. 6). The marked inhibition of arthritic disease progression by NBD-MCoTI-II persisted for the study duration. NBD-MCoTI-II was at least as effective as Pfizer's JAK 1/2 inhibitor CP690550 (Xeljanz™) and anti-TNF Ab control (see, Milici A J et al., (2008) Cartilage preservation by inhibition of Janus kinase 3 in two rodent models of rheumatoid arthritis, Arthritis Res Ther 10:R14), in ameliorating arthritic symptoms in the mouse CIA model. Finally, NBD-MCoTI-II also significantly (p<0.01) blocked the increase in paw thickness (FIG. 7) associated with CIA. The effect lasted the study duration.

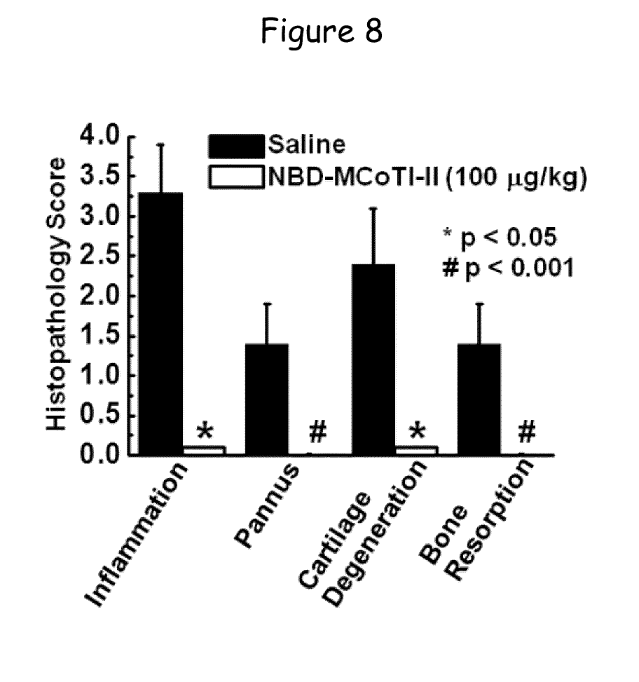

To further assess the efficacy of linear NBD-MCoTI-II we performed histopathology analysis of the studies on the joints of CIA mice treated with saline and NBD-MCoTI-II (100 μg/kg). Mice treated with saline displayed significant joint damage (FIG. 8, Panel A) as indicated by measurable scores for inflammation, pannus, cartilage damage and bone resorption (FIG. 8, Panel B). In marked contrast treatment of CIA mice with NBD-MCoTI-II (100 μg/kg) prevented joint damage with histopathology scores at or near zero (FIG. 8, Panels A and B).

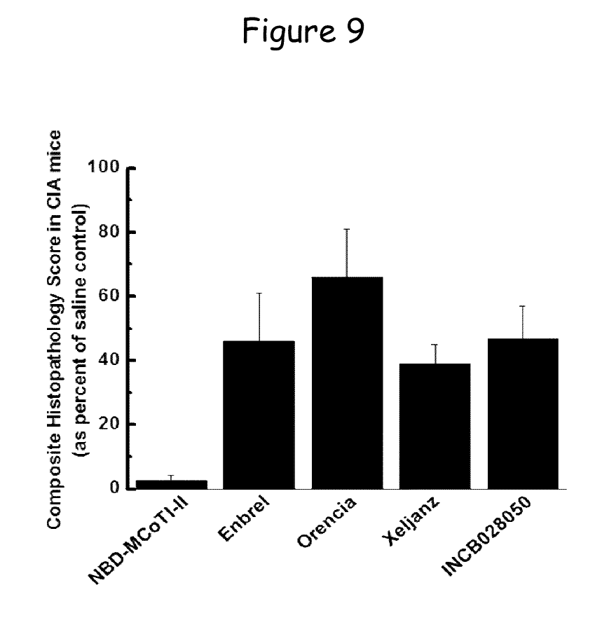

To compare the efficacy of NBD-MCoTI-II to existing anti-arthritic drugs we calculated a composite histopathology score for the CIA mice from the individual scores of inflammation, pannus, cartilage damage and bone resorption by adding them. Shown in FIG. 9 is a comparison of the composite histopathology scores for NBD-MCoTI-II (100 μg/kg) to published values for the existing anti-arthritic drugs Enbrel™ (15 mg/kg), Orencia™ (5 mg/kg), Xeljanz™ (10 mg/kg) and INCB028050 (15/mg). Data are presented as a percent of the composite histopathology scores for CIA mice treated with saline. FIG. 9 shows that NBD-MCoTI-II exhibits a remarkable reduction in composite histopathology scores as compared to existing anti-arthritic drugs (data for Enbrel™, Orenica™, Xeljanz™ and INCB028050 as shown in, e.g., Seeuws S. et al., (2010) A multiparameter approach to monitor disease activity in collagen-induced arthritis. Arthritis Res Ther 12:R160; Milici A J et al., (2008) Cartilage preservation by inhibition of Janus kinase 3 in two rodent models of rheumatoid arthritis. Arthritis Res Ther 10:R14; and Fridman J S et al., (2010) Selective inhibition of JAK1 and JAK2 is efficacious in rodent models of arthritis: preclinical characterization of INCB028050. J Immunol 184:5298-307, the contents of each of which are herein incorporated by reference in their entireties). NBD-MCoTI-II had a composite histopathology score almost zero while the existing anti-arthritic drugs were only capable of reducing the composite histopathology scores ˜40-60%. Thus, linear NBD-MCoTI-II prevented joint damage while the other anti-arthritic drugs were only capable of reducing joint damage.

Splenomegaly is associated with arthritis and has been observed in the mouse CIA model [(see, e.g., Foell J L et al., (2004) Engagement of the CD137 (4-1BB) costimulatory molecule inhibits and reverses the autoimmune process in collagen-induced arthritis and establishes lasting disease resistance. Immunology 113:89-98; and Liu Y et al., (2011) Therapeutic effects of TACI-Ig on collagen-induced arthritis by regulating T and B lymphocytes function in DBA/1 mice. Eur J Pharmacol 654:304-14); the contents of each of which are herein incorporated by reference in their entirities)]. In our study, saline-treated CIA mice had enlarged spleens with an average weight of 135±27 mg (normal spleen weight in male DBA/1J mice is ˜80 mg [(see, e.g., Peterson S et al., (2011) Effects of Portabella mushrooms on collagen-induced arthritis, inflammatory cytokines, and body composition in dilute brown non-agouti (DBA1) mice. Functional Foods in Health and Disease 1:279-296), the contents of which are herein incorporated by reference in its entirety]. NBD-MCoTI-II treatment significantly (p<0.01) reduced the spleen weight to 100±17 mg.

NBD-MCoTI-II displayed no obvious signs of toxicity or distress. Mouse body weights were unchanged relative to saline controls. Laboratory blood analysis including general blood chemistry, kidney function tests, liver enzyme levels (ALT, AST) and blood cell counts were within normal ranges. Kidney, liver and intestine weights were within normal ranges and no differences were observed between saline and NBD-MCoTI-II-treated CIA mouse.

Although the present invention has been described in considerable detail with reference to certain preferred embodiments, other embodiments are possible. The steps disclosed for the present methods, for example, are not intended to be limiting nor are they intended to indicate that each step is necessarily essential to the method, but instead are exemplary steps only. Therefore, the scope of the appended claims should not be limited to the description of preferred embodiments contained in this disclosure.

Recitation of value ranges herein is merely intended to serve as a shorthand method for referring individually to each separate value falling within the range. Unless otherwise indicated herein, each individual value is incorporated into the specification as if it were individually recited herein. All references cited herein are incorporated by reference in their entirety.

What is claimed is:

1. A fusion protein compromising NBD or a fragment thereof and MCoTI-I/II or a fragment thereof.

2. The fusion protein of claim 1, wherein the NBD comprises the amino acid sequence of SEQ ID NO:22.

3. The fusion protein of claim 1, wherein the NBD comprises the amino acid sequence of SEQ ID NO:23.

4. The fusion protein of claim 1, wherein the fusion protein is formed by replacing loop six of MCoTI-I/II with NBD or a fragment thereof.

5. The fusion protein of claim 1, wherein the NBD has the sequence of SEQ ID NO:4.

6. The fusion protein of claim 1, wherein the fusion protein is a linear molecule.

7. The fusion protein of claim 1, wherein the NBD is fused to the N-terminus of MCoTI-I/II.

8. The fusion protein of claim 7, wherein the fusion protein has the sequence represented by SEQ ID NO:10.

9. The fusion protein of claim 1, wherein the NBD is fused to the C-terminus of MCoTI-I/II.

10. The fusion protein of claim 1, wherein the fusion protein is a cyclic peptide.

11. The fusion protein of claim 1, wherein the fusion protein is represented by SEQ ID NO:9.

12. The fusion protein of claim 1, wherein the fusion protein is represented by a sequence selected from the group consisting of any of the sequences of SEQ ID NOS:13-21 and 28-44.

13. The fusion protein of claim 1, wherein the fusion protein compromises one or more linkers.

14. The fusion protein of claim 1, wherein the fusion protein further compromises the sequence of SEQ ID NO:45.

15. A pharmaceutical composition which comprises a fusion protein according claim 1.

16. A method of treating an inflammatory disease in a subject, comprising the step of administering a pharmaceutical composition which comprises a fusion protein according claim 1.

17. The method of claim 16, wherein the medical condition is due to at least in part to activation of nuclear factor kB (NF-κβ).

18. The method of claim 16, wherein the medical condition is selected from the group consisting of graft vs. host disease, sepsis, muscular dystrophy, cachexia, cardiac hypertrophy, cardiac ischemia, reperfusion injury, spinal cord injury, and cancer.
